(19) United States
(12) Patent Application Publication
(10) Pub. No.: US 2026/0346270 A1
(43) Pub. Date: Nov. 5, 2026

(54) STRAINER INSERT FOR MATRIX SEPARATION WITHIN A CENTRIFUGE TUBE

(71) Applicant: Board of Regents of the University of Nebraska, Lincoln, NE (US)

(72) Inventors: Andy Schnaubelt, Papillion, NE (US); M. Jana Broadhurst, Omaha, NE (US); David Brett-Major, Omaha, NE (US)

(21) Appl. No.: 19/099,340

(22) PCT Filed: Aug. 17, 2023
(86) PCT No.: PCT/US2023/072357

§ 371 Date: Jan. 28, 2025

Related U.S. Application Data
(60) Provisional application No. 63/398,645, filed on Aug. 17, 2022.

(51) Int. Cl. B04B 7/16; B04B 5/04; C12Q 1/6806

(52) U.S. Cl. CPC B04B 7/16; B04B 5/0414; C12Q 1/6806

(57) ABSTRACT

A strainer insert for matrix separation within a centrifuge tube is disclosed. In embodiments, the strainer insert includes: a platform configured to hold a sample collection matrix at an elevation within the centrifuge tube; a base structure configured to support the platform within the centrifuge tube; and a vertical member configured to extend from the platform toward a top opening of the centrifuge tube. The platform includes a plurality of openings configured to pass through sample material that is separated from the sample collection matrix during a centrifugation process. The vertical member is graspable to remove the strainer insert from the centrifuge tube after the centrifugation process, thereby allowing for removal of the sample collection matrix while the sample material remains within the centrifuge tube.

### CROSS-REFERENCE TO RELATED APPLICATIONS

The present application claims priority under 35 U.S.C. § 119(e) to U.S. Provisional Application Ser. No. 63/398,645, filed Aug. 17, 2022, and titled “Strainer Insert for Matrix Separation (SIMSep),” which is incorporated herein by reference in its entirety.

### TECHNICAL FIELD

The present disclosure generally relates to laboratory test equipment, more particularly to apparatus, systems, and methods for maintaining separation between a sample collection matrix and isolated sample material during and after centrifugation.

### BACKGROUND

When sampling individuals or environments for the presence of biologic, chemical, or other agents of health interest, there are few non-invasive specimen collection methods that consistently yield valid and reliable laboratory test results. A useful and under-utilized sampling method is to use a sample collection matrix, such as a wetted, sterile wipe or similarly functioning matrix. After sample collection, a challenge then exists in being able to separate material of interest from the sample collection matrix.

Common laboratory testing methods for pathogen detection and identification, for instance, require that the pathogen of interest be separated and/or isolated from the original sample collection matrix (e.g., a handwipe). Centrifugation is a near ubiquitous laboratory method used to separate various components of a fluid based on differing densities, and to separate liquids from solids. In the handwipe example, centrifugation is an appropriate method to separate the liquid fraction of the solution wetting the wipe, from the wipe itself. However, there are currently no commercial products that maintain separation between the sample collection matrix and the isolated sample material after completion of the centrifugation process. This presents a problem because the sample collection matrix may re-mix with and/or reabsorb the sample material.

Given the state of the art, there is a need for apparatus, systems, and methods for maintaining separation between a sample collection matrix and isolated sample material during and after centrifugation.

### SUMMARY

A strainer insert for matrix separation within a centrifuge tube is disclosed. The strainer insert is configured to retain a sample collection matrix at an elevation within the centrifuge tube while a centrifugation process is completed to separate sample material from the sample collection matrix. After the centrifugation process, the strainer insert prevents the sample collection matrix from re-mixing with and/or reabsorbing the sample material.

In embodiments, the strainer insert includes: a platform configured to hold a sample collection matrix at an elevation within the centrifuge tube; a base structure configured to support the platform within the centrifuge tube; and a vertical member configured to extend from the platform toward a top opening of the centrifuge tube. The platform includes a plurality of openings configured to pass through sample material that is separated from the sample collection matrix during a centrifugation process. The vertical member is graspable to remove the strainer insert from the centrifuge tube (e.g., by pulling up on the vertical member) after the centrifugation process, thereby allowing for removal of the sample collection matrix while the sample material remains within the centrifuge tube.

This Summary is provided solely as an introduction to subject matter that is fully described in the Detailed Description and Drawings. The Summary should not be considered to describe essential features nor be used to determine the scope of the Claims. Moreover, it is to be understood that both the foregoing Summary and the following Detailed Description are example and explanatory only and are not necessarily restrictive of the subject matter claimed.

### BRIEF DESCRIPTION OF THE DRAWINGS

The Detailed Description is provided with reference to the accompanying Drawings. The use of the same reference numbers in different instances in the Detailed Description and the Drawings may indicate similar or identical items. The Drawings are not necessarily to scale, and any disclosed processes may be performed in an arbitrary order, unless a certain order of steps/operations is inherent or specified in the Detailed Description or in the Claims.

FIG. 1 is a perspective view of a strainer insert for matrix separation, in accordance with one or more embodiments of this disclosure.

FIG. 2 is a perspective view of the strainer insert of FIG. 1 within a centrifuge tube, in accordance with one or more embodiments of this disclosure.

FIG. 3 is a side view of the strainer insert of FIG. 1, in accordance with one or more embodiments of this disclosure.

FIG. 4 is a rear view of the strainer insert of FIG. 1, in accordance with one or more embodiments of this disclosure.

FIG. 5 is a top view of the strainer insert of FIG. 1, in accordance with one or more embodiments of this disclosure.

FIG. 6 is a bottom view of the strainer insert of FIG. 1, in accordance with one or more embodiments of this disclosure.

FIG. 7 is a perspective view of a strainer insert for matrix separation, in accordance with one or more embodiments of this disclosure.

FIG. 8 is a perspective view of a strainer insert for matrix separation, in accordance with one or more embodiments of this disclosure.

FIG. 9 is a perspective view of a strainer insert for matrix separation, in accordance with one or more embodiments of this disclosure.

FIG. 10 is a perspective view of a strainer insert for matrix separation, in accordance with one or more embodiments of this disclosure.

FIG. 11 is a bottom view of the strainer insert of FIG. 10, in accordance with one or more embodiments of this disclosure.

FIG. 12 is another perspective view of the strainer insert of FIG. 10, in accordance with one or more embodiments of this disclosure.

FIG. 13 is a flow diagram illustrating a first portion of a method of sample processing for viral/pathogen detection, in accordance with one or more embodiments of this disclosure.

FIG. 14 is a flow diagram illustrating a second portion of the method of sample processing for viral/pathogen detection, in accordance with one or more embodiments of this disclosure.

### DETAILED DESCRIPTION

The present disclosure is directed to a strainer insert for matrix separation within a centrifuge tube and to a system and method associated therewith.

When sampling individuals or environments for the presence of biologic, chemical, or other agents of health interest, a useful and under-utilized sampling method is to use a sample collection matrix, such as a wetted, sterile wipe or similarly functioning matrix (e.g., any wipe, pad, gauze, cotton ball, sponge, or other absorbent/abrasive material). After collecting sample material by rubbing or wiping the sample collection matrix across a surface to absorb, abrase, or otherwise remove and capture sample material from the surface, a centrifugation process can be performed to separate the sample material (liquid and/or solid) from the sample collection matrix. However, there are currently no commercial products to keep the sample collection matrix away from liquid fractions that are separated from the sample collection matrix during centrifugation so that both remain separated after completion of the centrifugation process. The strainer insert solves this problem and allows for the sample collection matrix to be discarded and for the liquid fractions (including the isolated sample material) to be further processed for pathogen detection or any other type of testing.

The strainer insert is configured to retain a sample collection matrix at an elevation within a centrifuge tube while a centrifugation process is completed to separate sample material from the sample collection matrix. After the centrifugation process, the strainer insert prevents the sample collection matrix from re-mixing with and/or reabsorbing the sample material. The strainer insert is designed to hold the sample collection matrix above liquids/solids that separate from the sample collection matrix and move to a lower portion of a centrifuge tube during centrifugation so that: (1) the sample collection matrix and the separated sample material do not re-mix when centrifugation is complete; and (2) the strainer insert and the sample collection matrix can be easily removed in a single step that minimizes the potential for contamination of the sample material (e.g., by introduction of exogenous agents) and also minimizes the risk of exposing an individual performing the separation to harmful pathogens or chemicals.

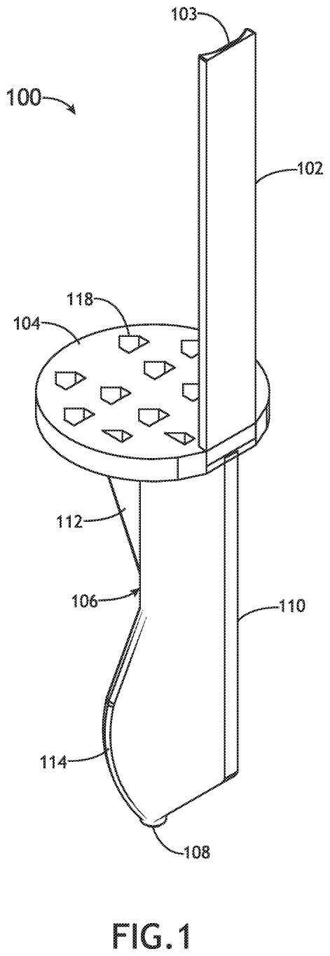

Example embodiments of the strainer insert are discussed below with reference to FIGS. 1 through 12. Those skilled in the art will appreciate that the embodiments illustrated in FIGS. 1 through 12 are specific examples that can be modified by changing dimensions or adding/removing non-essential components or fillers. Furthermore, two or more embodiments (or portions thereof) can be combined to achieve an additional embodiment that is not necessarily shown or described with reference to one drawing. As such, the drawings should not be construed as restrictive of any particular embodiment and are intended instead as visual aids to help describe configurations of certain components that may apply to multiple embodiments of the strainer insert.

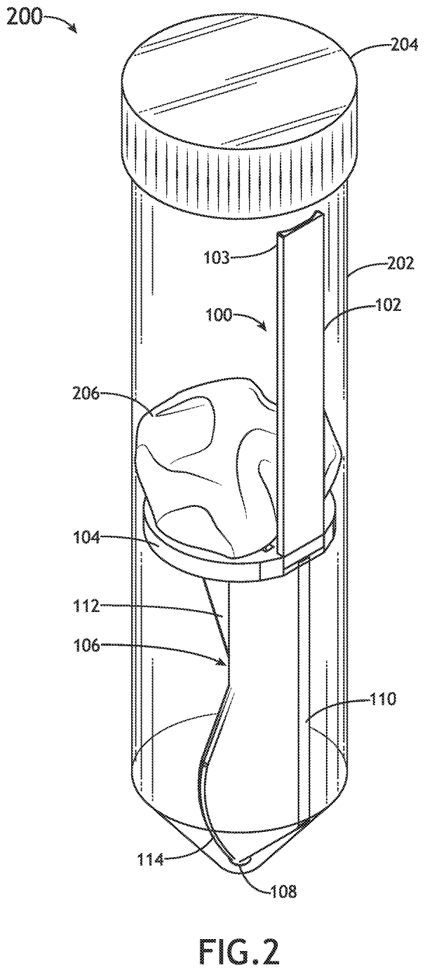

FIG. 1 illustrates a strainer insert 100 in accordance with an embodiment of this disclosure, and FIG. 2 illustrates a system 200 (e.g., a testing kit) that includes the strainer insert 100 disposed within a centrifuge tube 202. In some embodiments, the centrifuge tube 202 may have a substantially cylindrical body with a tapered bottom. The centrifuge tube 202 may also have a cap 204 configured to enclose a top opening of the centrifuge tube 202 to seal contents (e.g., sample collection matrix, sample material, etc.) within the centrifuge tube 202.

As shown in FIG. 2, the strainer insert 100 includes a platform 104 configured to hold a sample collection matrix 206 at an elevation within the centrifuge tube 202. The platform 104 is coupled to a base structure 106 that is configured to support the platform 104 within the centrifuge tube 202. In preferred embodiments, the base structure 106 is configured to hold the platform 104 at a fixed height within the centrifuge tube 202. The platform 104 may be coupled to the base structure 106 in any number of ways; for example, the platform 104 may be adhered, fused, or fastened to the base structure 106, connected by a friction-fit (e.g., snap-fit) coupling between the two components, or the platform 104 and the base structure 106 can be portions of a common 3D printed, injection molded, metal cast, or otherwise manufactured structure.

In embodiments, the base structure 106 includes a post 108 configured to extend from the platform 104 to a bottom of the centrifuge tube 202. In some embodiments, the post 108 is a thicker portion or rod-like portion of the base structure 106. Alternatively, the post 108 may simply be any longitudinal portion of the base structure 106 that extends from the platform 104 to the bottom of the centrifuge tube 202. In some embodiments, the post 108 extends about 40 to 60 millimeters below the platform 104 (e.g., approximately 54.8 millimeters in an example embodiment) and has a thickness/diameter of about 1 to 5 millimeters (e.g., approximately 2.85 millimeters in an example embodiment).

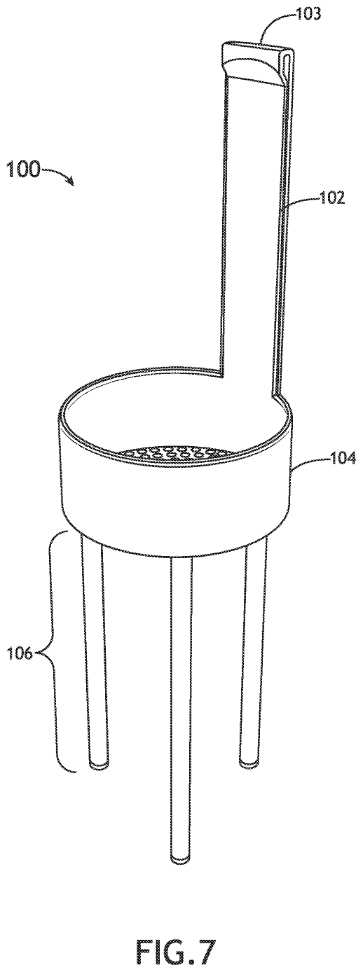

The post 108 may form a spine for blades, fins, and/or supports that extend outwardly from the post 108. The post 108 is preferably aligned with a central axis of the platform 104. However, in other embodiments, the post 108 may be off-center. For example, FIG. 7 illustrates an embodiment wherein the base structure 106 is formed by a plurality of off-center posts distributed evenly about the outer perimeter of the platform 104.

Referring again to FIGS. 1 and 2, the base structure 106 may further include a blade 110 that extends from the post 108 toward an outer perimeter of the platform 104. In some embodiments, the blade 110 extends all the way (or most of the way) from the post 108 to the outer perimeter (or edge) of the platform 104. The blade 110 may have an outer edge that conforms to an inner surface of the centrifuge tube 202. For example, the outer edge of the blade 110 may be configured to extend along the inner surface of the centrifuge tube 202 from the platform 104 all the way down to the bottom, or near the bottom, of the centrifuge tube 202. In some embodiments, the blade 110 may have a substantially trapezoidal profile with a tapered blade section that generally conforms (e.g., in its edge shape and tapered profile) to the tapered bottom portion of the centrifuge tube 202.

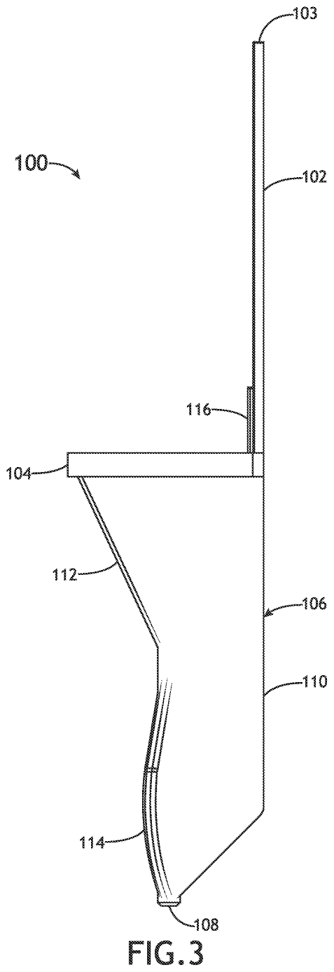

As shown in FIG. 3, the base structure 106 may further include a diagonal support 112 between the platform 104 and an upper section (e.g., upper half) of the post 108. In some embodiments, the diagonal support 112 may have a substantially triangular profile with one side contacting the post 108, one side contacting the platform 104, and one side extending between the post 108 and the platform 104. The diagonal support 112 may extend from the post 108 toward the outer perimeter of the platform 104 in a direction opposite that of the blade 110. For example, the diagonal support 112 and the blade 110 may be separated by an angle of about 180 degrees, as shown in FIG. 6. In some embodiments, the diagonal support 112 extends all the way (or most of the way) from the post 108 to the outer perimeter (or edge) of the platform 104.

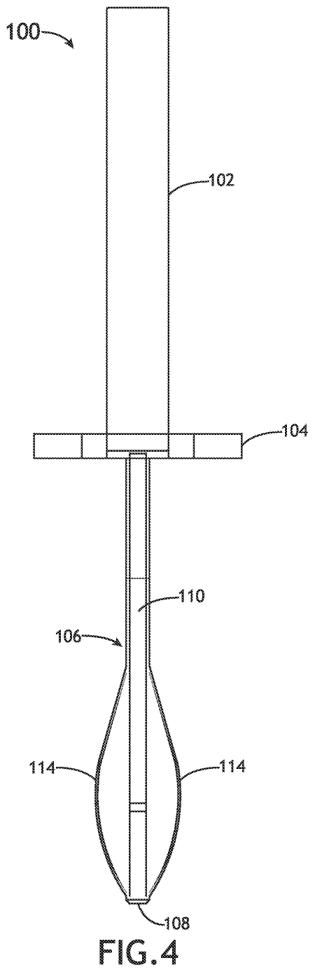

The base structure 106 may also include one or more fins 114 extending outwardly from a lower section (e.g., lower half) of the post 108. The base structure 106 preferably includes at least two fins 114, as shown in FIG. 4. In some embodiments, each of the fins 114 has a half-teardrop profile. In some embodiments, each fin 114 extends about 25 to 30 millimeters along the lower section of the post 108 (e.g., approximately 28.4 millimeters in an example embodiment).

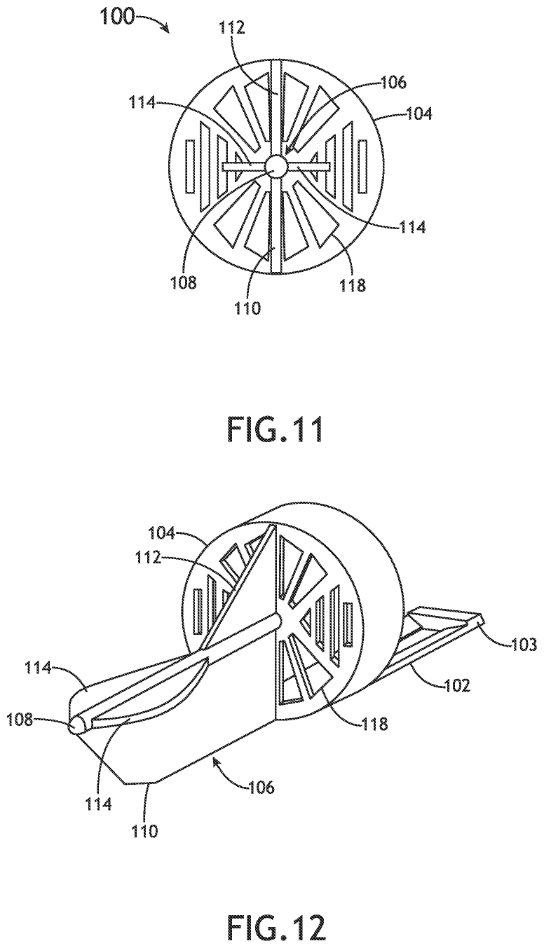

The two fins 114 may be located on opposite sides of the diagonal support 112 or opposite sides of the blade 110. In some embodiments, each of the two fins 114 is at an angle in the range of 45 to 90 degrees with respect to the diagonal support 112. For example, each of the fins 114 may be separated from a respective side surface of the diagonal support 112 by an angle of about 60 degrees as shown in FIG. 6 (bottom view), or an angle of about 90 degrees as shown in FIG. 11 (bottom view).

In some embodiments, the blade 110, diagonal support 112, and/or fins 114 may include holes, slots, slits, or other voids to reduce drag. This may help prevent deformation of the blade 110, diagonal support 112, and/or fins 114 during centrifugation.

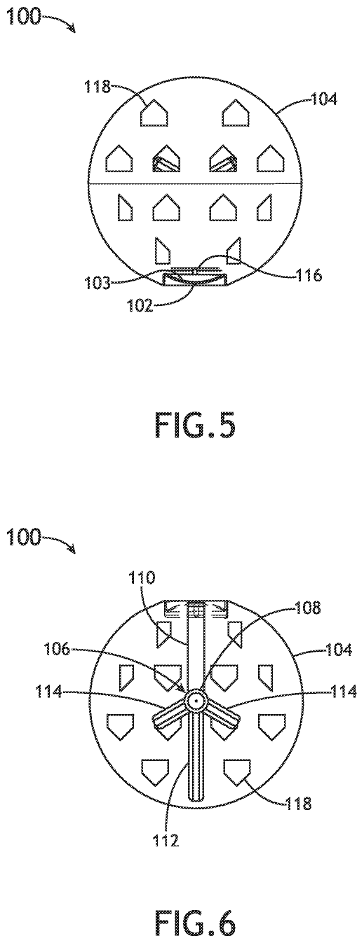

Referring again to FIGS. 1 and 2, the platform 104 includes a plurality of openings 118 configured to pass through sample material that is separated from the sample collection matrix 206 during a centrifugation process. For example, during the centrifugation process, at least a portion of the collected sample material, and possibly other liquids or small solids, will fall through the openings 118 in the platform 104 into a lower portion of the centrifuge tube 202 (i.e., the portion of the centrifuge tube 202 below the platform 104). In some embodiments, most of the openings have a width of about 1 to 5 millimeters (e.g., approximately 3.1 millimeters in an example embodiment) and a length of about 1 to 5 millimeters (e.g., approximately 3.1 millimeters in an example embodiment). In FIGS. 5 and 6, the openings 118 are shown as mostly pentagon-shaped openings; however, any other shape may be appropriate (e.g., circles, semi-circles, slits, slots, polygons, asterisks, etc.) for the openings 118.

In embodiments, the platform 104 has a circular or substantially circular outer perimeter. Alternatively, the platform 104 may have an outer perimeter that defines a different shape (e.g., ellipse, polygon, etc.) or a combination of shapes (e.g., partially polygonal and partially circular, or any other combination). In some embodiments, such as the various alternative embodiments illustrated in FIGS. 7 through 12, the platform 104 may be surrounded by an outer wall to form a basket-like structure that is configured to hold the sample collection matrix 206.

The platform 104 is shown as being substantially planar/flat in FIGS. 1 through 12. However, in other embodiments, the platform 104 may have a concave or convex surface for supporting the sample collection matrix 206. In some embodiments, the platform 104 has a thickness of about 1 to 5 millimeters (e.g., approximately 3 millimeters in an example embodiment) and a diameter of about 20 to 30 millimeters (e.g., approximately 25.6 millimeters in an example embodiment).

The platform 104 is coupled to the base structure 106 that is configured to support the platform 104 within the centrifuge tube 202. The platform 104 is also coupled to (e.g., adhered to, fused to, otherwise attached/connected to, or structurally integrated with) a vertical member 102 that is configured to extend from the platform 104 toward the top opening of the centrifuge tube 202. In some embodiments, the vertical member 102 extends about 40 to 60 millimeters above the platform 104 (e.g., approximately 52.5 millimeters in an example embodiment) and has a thickness of about 1 to 2 millimeters (e.g., approximately 1.4 millimeters in an example embodiment).

The vertical member 102 is graspable to remove the strainer insert 100 from the centrifuge tube 202 after the centrifugation process, thereby allowing for removal of the sample collection matrix 206 while any sample material that was separated from the sample collection matrix 206 remains within the centrifuge tube 202. That is, the sample material and any other liquids or small solids that fell through the openings 118 in the platform 104 into the lower portion of the centrifuge tube 202 during the centrifugation process will remain in the centrifuge tube 202 after the strainer insert 100 is taken out to remove the sample collection matrix 206 from the centrifuge tube 202. The strainer insert 100 is easily removed by pulling up on the vertical member 102. In some embodiments, the vertical member 102 includes a tab 103 at its top end for improved grip and/or maneuverability.

The vertical member 102 may be configured to extend along an inner surface of the centrifuge tube 202, as shown in FIG. 2. As a result, the vertical member 102 may help stabilize the platform 104 while the centrifuge tube 202 is agitated during the centrifugation process. In some embodiments, the vertical member 102 and the blade 110 of the base structure 106 are aligned. For example, the vertical member 102 may be axially aligned with the outer edge of the blade 110 such that the vertical member 102 and the outer edge of the blade 110 extend colinearly along the inner surface of the centrifuge tube 202. This configuration may provide improved stability for the strainer insert 100 during centrifugation.

As shown in FIG. 3, the vertical member 102 may also be reinforced by a support structure 116 (e.g., a supportive fin or additional layer(s) of material) at a base of the vertical member 102, that is, at the interface between the vertical member 102 and the platform 104. The support structure 116 is also shown in FIG. 5 (top view). The support structure 116 at the base of the vertical member 102 helps to prevent deformation of the vertical member 102 during centrifugation. As further shown in FIG. 5, the vertical member 102 itself may have a curved (e.g., concave) inner surface to improve its structural integrity, with or without the additional support structure 116.

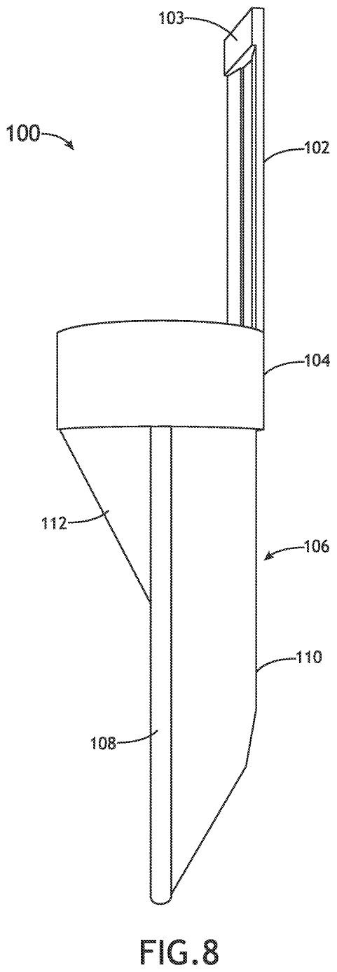

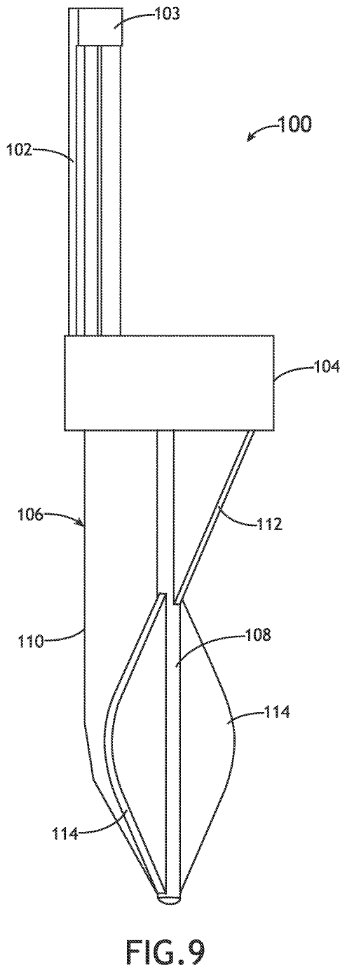

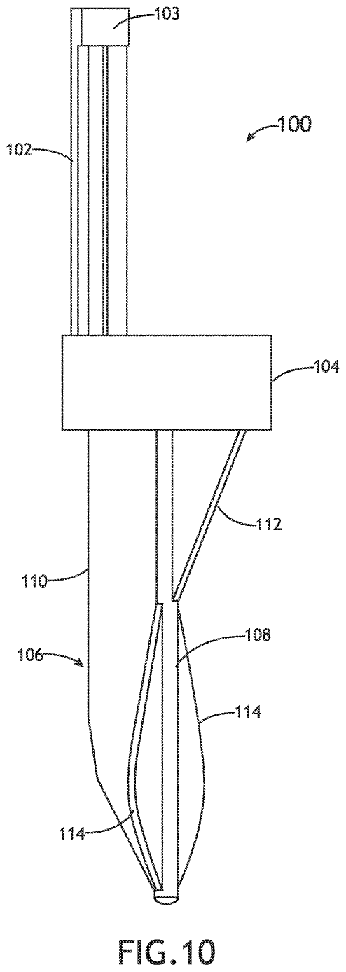

Alternative embodiments of the strainer insert 100 are illustrated in FIGS. 7 through 12. In particular, FIG. 7 illustrates an alternative embodiment of the strainer insert 100 wherein the base structure 106 is comprised of a plurality of posts distributed about the perimeter of the platform 104, but without any blades, fins, or diagonal supports; FIG. 8 illustrates an alternative embodiment of the strainer insert 100 wherein the base structure 106 is comprised of the post 108, blade 110, and diagonal support 112 in a similar construction to FIGS. 1 through 6, but without any fins; FIG. 9 illustrates an alternative embodiment of the strainer insert 100 wherein the base structure 106 is comprised of the post 108, blade 110, diagonal support 112, and fins 114 in a similar construction to FIGS. 1 through 6, but with differently shaped fins 114 (e.g., fins 114 having a substantially triangular profile with a curved outer corner) that are substantially orthogonal to the diagonal support 112; and FIGS. 10 through 12 illustrate an alternative embodiment of the strainer insert 100 wherein the base structure 106 is comprised of the post 108, blade 110, diagonal support 112, and fins 114 in a similar construction to FIGS. 1 through 6, but with fins 114 that are substantially orthogonal to the diagonal support 112.

The embodiments in FIGS. 7 through 12 also differ from FIGS. 1 through 6 by including an outer wall that surrounds the platform 104 to form a basket-like structure, differently shaped openings 118 in the platform 104, and may contain other differences in the overall shape, structure, or appearance. Nevertheless, as previously noted herein, two or more embodiments (or portions thereof) can be combined to achieve an additional embodiment of the strainer insert 100 that is not necessarily shown or described with reference to one drawing. As such, the drawings should not be construed as restrictive of any particular embodiment and are intended instead as visual aids to help describe configurations of certain components that may apply to multiple embodiments of the strainer insert 100.

The strainer insert 100 may be manufactured from durable, inert, thermostable polymers such as medical grade polypropylene or polycarbonate, although other materials may be utilized depending on the requirements of any particular laboratory/testing application. In some embodiments, the strainer insert 100 is formed by a single mold/print. For example, the strainer insert 100 may be entirely formed by a continuous 3D printed or injection-molded structure. Alternatively, portions of the strainer insert 100 may be separately printed, molded, or cast and then assembled and/or fused together. For example, the vertical member 102, the platform 104, the base structure 106, or any portion of a component, may be printed, molded, or cast separately and then assembled and/or fused together to create the final device. Separately manufacturing portions of the strainer insert 100 may have advantages for injection molding processes because simpler molds and/or less material can be utilized.

A method of sample collection and processing may employ a strainer insert 100 and system 200 as described herein. The method may include the following steps: (1) collecting sample material from a surface by rubbing or wiping a sample collection matrix 206 across the surface; (2) placing the sample collection matrix 206 within a centrifuge tube 202 using the strainer insert 100, wherein the platform 104 is configured to hold the sample collection matrix 206 at an elevation within the centrifuge tube 202; (3) performing a centrifugation process to separate the sample material from the sample collection matrix 206, wherein the separated sample material is passed through the openings 118 in the platform 104 of the strainer insert 100 to a lower portion of the centrifuge tube 202; and (4) removing the strainer insert 100 from the centrifuge tube 202 by pulling up on the vertical member 102 of the strainer insert 100 so that the sample collection matrix 206 is removed with the strainer insert 100 while the sample material remains within the centrifuge tube 202. In some embodiments, at step (4), the sample collection matrix 206 may be removed by itself before taking out the strainer insert 100. For example, the sample collection matrix 206 can be grasped from the platform 104 of the strainer insert 100 and discarded before the strainer insert 100 is pulled from the centrifuge tube 202. The foregoing method may further include any other step or process that is required for centrifugation or necessitated by the strainer insert 100 or system 200 described herein.

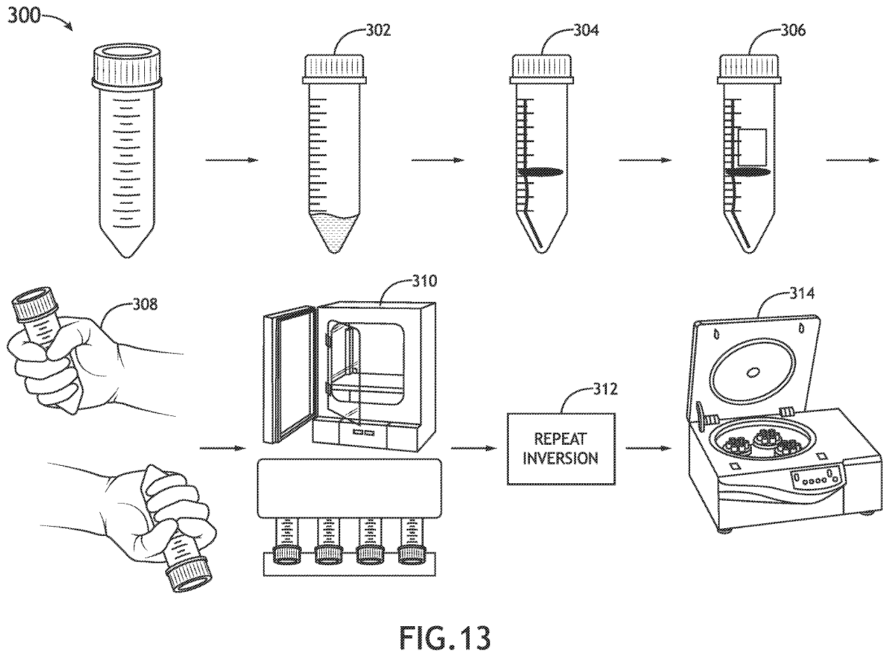

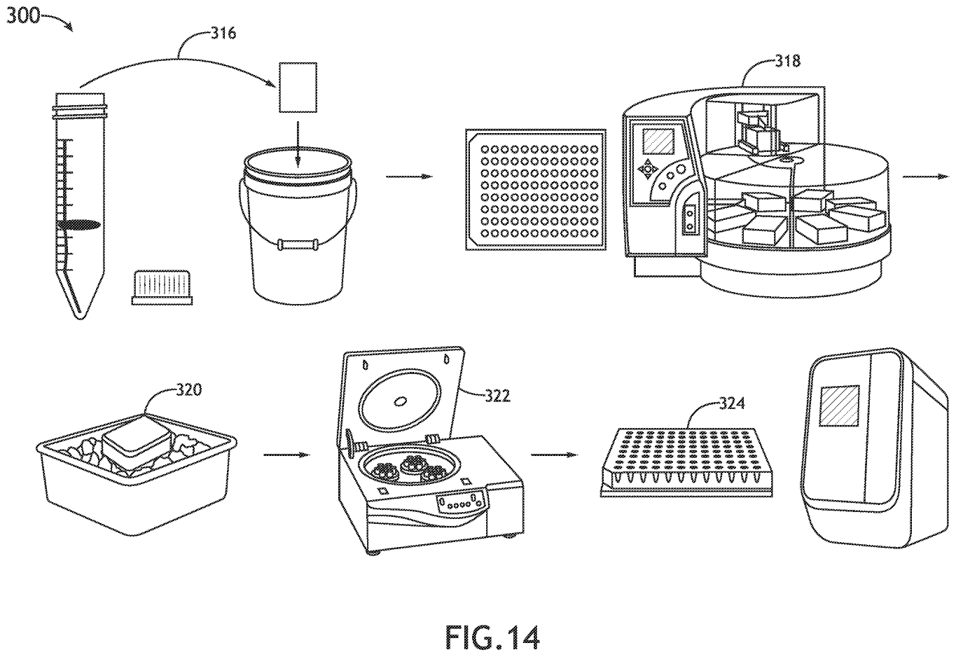

FIGS. 13 and 14 illustrate a more specific method 300 of sample processing for viral/pathogen detection. The method 300 also employs a strainer insert 100 and system 200 as described herein. In addition to the steps described below, the method 300 may further include any other step or process that is required for centrifugation or necessitated by the strainer insert 100 or system 200 described herein.

FIG. 13 illustrates a first part of the method 300, wherein pathogen separation steps are performed after collecting sample material from a surface by rubbing or wiping a sample collection matrix 206 across the surface. At step 302 of the method 300, a processing solution is added to the centrifuge tube 202. For example, 2 mL of 5% sodium dodecyl sulfate (SDS) solution may be added to a 50 mL centrifuge tube. At step 304 of the method 300, the strainer insert 100 is placed within the centrifuge tube 202. At step 306 of the method 300, the sample collection matrix 206 is placed upon the platform 104 of the strainer insert 100, within the centrifuge tube 202. For example, a collected alcohol prep specimen wipe may be disposed within the centrifuge tube 202 on the platform 104 of the strainer insert 100. At step 308 of the method 300, the centrifuge tube 202 is inverted a number of times so that the processing solution mixes with the sample collection matrix 206. For example, the centrifuge tube 202 may be inverted about 10 times or so. At step 310 of the method 300, the centrifuge tube 202 is placed within a tube rack having a lid, turned upside down (so that the centrifuge tube 202 is inverted) and incubated. In an example embodiment, the incubation is performed at a temperature of about 60 degrees Centigrade for about 15 minutes. At step 312 of the method 300, the centrifuge tube 202 is again inverted a number of times (as was done in step 308). For example, the centrifuge tube 202 may be inverted again about 10 times or so. At step 314 of the method 300, the tube 202 is centrifuged. For example, the tube 202 may be centrifuged for about 10 minutes at 1500×g.

FIG. 14 illustrates a second part of the method 300, wherein RNA extraction and reverse transcriptase-polymerase chain reaction (RT-PCR) testing steps are performed after the pathogen separation steps of FIG. 13. Following centrifugation, at step 316 of the method 300, the strainer insert 100 and sample collection matrix 206 are removed from the centrifuge tube 202. For example, the strainer insert 100 may be removed by pulling up on the vertical member 102 to withdraw the strainer insert 100, carrying the sample collection matrix 206 on its platform 104, from the centrifuge tube 202. In some embodiments, at step 316, the sample collection matrix 206 may be removed by itself before taking out the strainer insert 100. For example, the sample collection matrix 206 can be grasped from the platform 104 of the strainer insert 100 and discarded before the strainer insert 100 is pulled from the centrifuge tube 202. After removal, the strainer insert 100 and/or sample collection matrix 206 may be decontaminated in an antiviral solution (e.g., 10% bleach solution). During step 316, a handler should change gloves in between each centrifuge tube 202 to avoid cross-contamination. At step 318 of the method 300, an RNA sample is extracted by further processing the centrifuge tube 202 in an RNA extraction system. For example, about 200 microliters of RNA sample may be extracted using an extraction system, such as the KingFisher Flex, used with a viral/pathogen kit, such as the MagMax Viral/Pathogen Kit (A42352). At steps 320 and 322 of the method 300, the RNA sample may be further processed on an elution plate (on ice) and on a quick spin plate, respectively. At step 324 of the method 300, RT-PCR testing is performed on the RNA sample, for example, to test for the presence of a pathogen, such as SARS-COV-2 or Influenza A.

The foregoing steps may be performed for a plurality of centrifuge tubes 202 containing respective strainer inserts 100 and sample collection matrixes 206. In this regard, any of the steps (e.g., the incubation and centrifugation steps, extraction and testing steps, etc.) may be performed on a plurality of centrifuge tubes 202 at the same time. Furthermore, certain steps may be performed in parallel or in a different order than listed above, to the extent possible and unless otherwise specified in the claims.

Although the technology has been described with reference to the embodiments illustrated in the attached drawing figures, equivalents may be employed, and substitutions may be made herein without departing from the scope of the technology as recited in the claims. Components illustrated and described herein are examples of devices and components that may be used to implement the embodiments of the present invention and may be replaced with other devices and components without departing from the scope of the invention. Furthermore, any dimensions, degrees, and/or numerical ranges provided herein are to be understood as non-limiting examples unless otherwise specified in the claims.

1. A strainer insert for matrix separation within a centrifuge tube, comprising:
a platform configured to hold a sample collection matrix at an elevation within the centrifuge tube, the platform including a plurality of openings configured to pass through sample material that is separated from the sample collection matrix during a centrifugation process;
a base structure configured to support the platform within the centrifuge tube; and a vertical member configured to extend from the platform toward a top opening of the centrifuge tube, the vertical member being graspable to remove the strainer insert from the centrifuge tube.

2. The strainer insert of claim 1, wherein the platform has a circular or substantially circular outer perimeter.

3. The strainer insert of claim 1, wherein the platform is surrounded by an outer wall to form a basket-like structure.

4. The strainer insert of claim 1, wherein the base structure comprises a post configured to extend from the platform to a bottom of the centrifuge tube.

5. The strainer insert of claim 4, wherein the post is aligned with a central axis of the platform.

6. The strainer insert of claim 4, wherein the base structure further comprises a blade that extends from the post toward an outer perimeter of the platform.

7. The strainer insert of claim 6, wherein the blade has an outer edge that conforms to an inner surface of the centrifuge tube.

8. The strainer insert of claim 6, wherein the base structure further comprises a diagonal support between the platform and an upper section of the post, wherein the diagonal support extends from the post toward the outer perimeter of the platform in a direction opposite that of the blade.

9. The strainer insert of claim 8, wherein the base structure further comprises two fins extending outwardly from a lower section of the post, wherein the two fins are on opposite sides of the diagonal support, and each of the two fins is at an angle in the range of 45 to 90 degrees with respect to the diagonal support.

10. (canceled)

11. A system, comprising:
a sample collection matrix;
a centrifuge tube; and
strainer insert configured to be placed within the centrifuge tube, the strainer insert comprising:
a platform configured to hold the sample collection matrix at an elevation within the centrifuge tube, the platform including a plurality of openings configured to pass through sample material that is separated from the sample collection matrix during a centrifugation process;
a base structure configured to support the platform within the centrifuge tube; and a vertical member configured to extend from the platform toward a top opening of the centrifuge tube, the vertical member being graspable to remove the strainer insert from the centrifuge tube.

12. The system of claim 11, wherein the platform has a circular or substantially circular outer perimeter.

13. The system of claim 11, wherein the platform is surrounded by an outer wall to form a basket-like structure.

14. The system of claim 11, wherein the base structure comprises a post configured to extend from the platform to a bottom of the centrifuge tube.

15. The system of claim 14, wherein the post is aligned with a central axis of the platform.

16. The system of claim 14, wherein the base structure further comprises a blade that extends from the post toward an outer perimeter of the platform.

17. The system of claim 16, wherein the blade has an outer edge that conforms to an inner surface of the centrifuge tube.

18. The system of claim 16, wherein the base structure further comprises a diagonal support between the platform and an upper section of the post, wherein the diagonal support extends from the post toward the outer perimeter of the platform in a direction opposite that of the blade.

19. The system of claim 18, wherein the base structure further comprises two fins extending outwardly from a lower section of the post, wherein the two fins are on opposite sides of the diagonal support, and each of the two fins is at an angle in the range of 45 to 90 degrees with respect to the diagonal support.

20. (canceled)

21. (canceled)

22. A method of sample collection and processing, comprising:
collecting sample material from a surface by rubbing or wiping a sample collection matrix across the surface;
placing the sample collection matrix within a centrifuge tube using a strainer insert, the strainer insert including: a platform configured to hold the sample collection matrix at an elevation within the centrifuge tube; a base structure configured to support the platform within the centrifuge tube; and a vertical member configured to extend from the platform toward a top opening of the centrifuge tube;
performing a centrifugation process to separate the sample material from the sample collection matrix, wherein the sample material is passed through a plurality of openings in the platform of the strainer insert; and
removing the strainer insert from the centrifuge tube by pulling up on the vertical member of the strainer insert so that the sample collection matrix is removed with the strainer insert while the sample material remains within the centrifuge tube.

23. The method of claim 22, further comprising:
performing an RNA extraction process on the sample material remaining in the centrifuge tube after removing the strainer insert and the sample collection matrix from the centrifuge tube; and
performing a reverse transcriptase-polymerase chain reaction (RT-PCR) test on an RNA sample extracted from the sample material.
